(19) United States
(12) Patent Application Publication
(10) Pub. No.: US 2006/0188911 A1
(43) Pub. Date: Aug. 24, 2006

(54) Genetic testing method

(75) Inventors: Yasuhiro Otomo, Kobe (JP); Kazuki Nakabayashi, Kobe (JP); Motonari Daito, Kobe (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION

(21) Appl. No.: 11/354,945

(22) Filed: Feb. 16, 2006

(30) Foreign Application Priority Data
Feb. 18, 2005 (JP) 2005-043272

(51) Int. Cl. C12Q 1/68; C12P 19/34

(52) U.S. Cl. 435/6; 435/91.2

(57) ABSTRACT

A genetic testing method for specifically amplifying a DNA complementary to a target nucleic acid existing in a biologic sample, measuring the amplified DNA, and generating diagnosis support information on the basis of a measurement result is described that includes steps of: (a) specifically amplifying a DNA complementary to an internal standard nucleic acid by using a known amount of the internal standard nucleic acid in the absence of the biologic sample component, and obtaining a first measurement result on the basis of the amplification, wherein the DNA has a property to be specifically amplified in the presence of the biologic sample component; (b) specifically amplifying the DNA complementary to the target nucleic acid in the presence of the biologic sample component, and obtaining a second measurement result on the basis of the amplification; and (c) comparing the first and second measurement results to obtain diagnosis support information.

### TECHNICAL FIELD

The present invention relates to a genetic testing method for specifically amplifying a target nucleic acid existing in a biologic sample, measuring the amplified target nucleic acid, and generating diagnosis support information on the basis of a measurement result.

### BACKGROUND

In recent years, genetic testing is being rapidly spread in the field of clinical diagnosis. Genetic testing is conducted for clinical purposes to determine the presence/absence of a mutation, karyotype, and the like related to a hireditary disease by analyzing a nucleic acid, chromosome, or the like. An example of the genetic testing is diagnosis of metastasis of cancer to a lymph node. Cancer cells spread from the primary focus to all of the body via blood vessels and lymphatic vessels. In an operation of cancer, it is necessary to remove the focus as reliably as possible. Consequently, it is required to accurately detect metastasis and perform proper treatment in accordance with the degree of metastasis. Therefore, diagnosis of metastasis of cancer cells to lymph nodes during an operation is extremely significant. One of methods of diagnosis of metastasis of cancer to a lymph node is a method of detecting, as a target nucleic acid, a nucleic acid of protein which is hardly expressed or is expressed in a small amount in normal cells but often is expressed in cancer cells. Development in the gene analysis techniques in recent years realizes effective cancer diagnosis by amplifying a target nucleic acid included in a lymph node tissue removed from an organism and detecting the amplified target nucleic acid.

In the case of determining metastasis of cancer cells to lymph nodes by amplification and detection of a target nucleic acid as described above, usually, a lymph node is homogenized to extract a nucleic acid into a solution, a measurement sample obtained by purifying the nucleic acid in the solution is prepared, and the target nucleic acid in the measurement sample is amplified and detected. However, it takes long time to purify the nucleic acid, so that it takes time to obtain a quantitative result of the target nucleic acid by such a method and a problem occurs such that metastasis of cancer cannot be determined by promptly detecting the target nucleic acid. In diagnosis of metastasis of cancer cells to lymph nodes during an operation, the strategy of treatment in the operation is determined according to the result of determination of metastasis of cancer. Consequently, it is important to determine metastasis promptly.

From such a viewpoint, when a solution obtained by homogenizing lymph nodes without performing extraction and purification of nucleic acids or a supernatant of the solution is used as a measurement sample at the time of preparing the measurement sample, the target nucleic acid can be detected promptly. However, in the case of amplifying the nucleic acid by using such a measurement sample, as compared with the case of amplifying a nucleic acid by using a measurement sample prepared by purifying the nucleic acid, the amount of substances which inhibit, or suppress amplification of the nucleic acid (hereinafter referred to as “amplification inhibitors”) derived from lymph nodes is larger. The influence of the amplification inhibitors is very strong, and a problem such that a correct measurement value cannot be obtained occurs. In the case of preparing measurement samples from lymph nodes, there is also a problem that the degree of inhibition varies among samples.

A method is known which uses mRNA of a β actin gene as an internal standard substance at the time of detecting a target nucleic acid (mRNA) corresponding to a protein related to cancer. The method is disclosed in, for example, International Patent Publication No. WO 03/70935. By using mRNA of a housekeeping gene such as the β actin gene as an internal standard, relative detection of a target nucleic acid can be performed without considering the efficiency of extracting the target nucleic acid and the amplification efficiency of cDNA.

With respect to the β actin, however, since the original expression amount in the lymph node removed from a living body is not known, whether an amplification inhibitor in the lymph node exerts an influence on amplification of cDNA of the target nucleic acid (mRNA) or not may not be recognized even when mRNA of the housekeeping gene is used as an internal standard.

A nucleic acid measuring method is also known, for measuring a target nucleic acid by using at least one kind of a nucleic acid probe which is a nucleic acid probe made of a kind of oligo nucleotide marked with at least one kind of fluorescent dye (hereinbelow, simply called nucleic acid probe) and being hybridized to a corresponding nucleic acid (target nucleic acid), thereby changing the fluorescent character of the marked fluorescent dye. According to the method, a measurement system includes at least one kind of a target nucleic acid and at least one kind of an internal standard nucleic acid of a known amount corresponding to the amount of the target nucleic acid, and hybridization reaction and/or nucleic acid amplification reaction is allowed to occur by a reaction system including at least one kind of a nucleic probe (hereinbelow, target nucleic acid probe) made of oligo nucleotide specific to the target nucleic acid and marked with at least one kind of fluorescent dye, and/or a nucleic acid probe (hereinbelow, internal standard nucleic acid probe) specific to an internal standard nucleic acid and made of oligo nucleotide marked with at least one kind of fluorescent dye. A change or a change amount before and after hybridization, of the fluorescent character of the target nucleic acid probe caused by the hybridization between the target nucleic acid probe and the target nucleic acid, and a change or a change amount before and after hybridization, of the fluorescent character of the internal standard nucleic acid probe caused by the hybridization between the internal standard nucleic acid probe and the internal standard nucleic acid are measured with at least one kind of measurement wavelength. From the measurement value obtained and the addition amount of the internal standard nucleic acid, the target nucleic acid and/or the target nucleic acid before nucleic acid amplification reaction is/are measured. Such a technique is disclosed in, for example, Japanese Patent Laid-Open No. 2004-203.

At a site of actual diagnosis such as the above-described diagnosis of metastasis of cancer cells to lymph nodes during an operation, it is necessary to obtain the result promptly. In many cases, the density and the quantitative value of the target nucleic acid are used for reference. However, Japanese Patent Laid-Open No. 2004-203 does not consider such circumstances.

### SUMMARY

The scope of the present invention is defined solely by the appended claims, and is not affected to any degree by the statements within this summary.

A genetic testing method for specifically amplifying a DNA complementary to a target nucleic acid existing in a biologic sample, measuring the amplified DNA, and generating diagnosis support information on the basis of a measurement result, embodying features of the present invention includes: (a) specifically amplifying a DNA complementary to an internal standard nucleic acid by using a known amount of the internal standard nucleic acid in the absence of the biologic sample component, and obtaining a first measurement result on the basis of the amplification, wherein the DNA has a property to be specifically amplified in the presence of the biologic sample component; (b) specifically amplifying a DNA complementary to the internal standard nucleic acid by using an amount of a nucleic acid having a sequence identical to a sequence of the internal standard nucleic acid in the presence of the biologic sample component, and obtaining a second measurement result on the basis of the amplification, wherein the amount of the nucleic acid is the same as the known amount of the internal standard nucleic acid to be used in the step (a); (c) specifically amplifying the DNA complementary to the target nucleic acid in the presence of the biologic sample component, and obtaining a third measurement result on the basis of the amplification; (d) obtaining diagnosis support information on the basis of the third measurement result and a predetermined reference value; and (e) determining influence on the diagnosis support information of an amplification inhibitor in the biologic sample component on the basis of the first and second measurement results.

A second genetic testing method for specifically amplifying a DNA complementary to a target nucleic acid existing in a biologic sample, measuring the amplified DNA, and generating diagnosis support information on the basis of a measurement result, embodying features of the present invention includes: (a) specifically amplifying a DNA complementary to an internal standard nucleic acid by using a known amount of the internal standard nucleic acid in the absence of the biologic sample component, and obtaining a first measurement result on the basis of the amplification, wherein the DNA has a property to be specifically amplified in the presence of the biologic sample component; (b) specifically amplifying a DNA complementary to the internal standard nucleic acid by using an amount of a nucleic acid having a sequence identical to a sequence of the internal standard nucleic acid in the presence of the biologic sample component, and obtaining a second measurement result on the basis of the amplification, wherein the amount of the nucleic acid is the same as the known amount of the internal standard nucleic acid to be used in the step (a); (c) specifically amplifying the DNA complementary to the target nucleic acid in the presence of the biologic sample component, and obtaining a third measurement result on the basis of the amplification; (d) correcting either the third measurement result or a reference value corresponding to a reference target nucleic acid amount on the basis of first and second measurement results; and (e) obtaining diagnosis support information on the basis of the corrected one of the third measurement result and the reference value, and the other.

A third genetic testing method for specifically amplifying a DNA complementary to a target nucleic acid existing in a biologic sample, measuring the amplified DNA, and generating diagnosis support information on the basis of a measurement result, embodying features of the present invention includes: (a) specifically amplifying a DNA complementary to an internal standard nucleic acid by using a known amount of the internal standard nucleic acid in the absence of the biologic sample component, and obtaining a first measurement result on the basis of the amplification, wherein the DNA has a property to be specifically amplified in the presence of the biologic sample component; (b) specifically amplifying the DNA complementary to the target nucleic acid in the presence of the biologic sample component, and obtaining a second measurement result on the basis of the amplification; and (c) comparing the first and second measurement results to obtain diagnosis support information.

### BRIEF DESCRIPTION OF THE DRAWINGS

FIG. 1 is a graph showing the influence of amplification inhibition of an amplification inhibitor that inhibits amplification of LAMP of a target nucleic acid and an internal standard nucleic acid.

FIG. 2 is a graph showing the relation between time required to detect a target nucleic acid in a sample which is not diluted and time required to detect a target nucleic acid in a diluted sample.

FIG. 3 is a diagram showing the relation between amplification of a target nucleic acid having density as a reference of determination and amplification of an internal standard nucleic acid having density corresponding to the reference.

FIG. 4 is a graph showing the relation between amplification of an internal standard nucleic acid having density corresponding to the reference of determination and amplification of a target nucleic acid having density at which metastasis of cancer to a lymph node is strongly positive.

FIG. 5 is a graph showing the relation between amplification of an internal standard nucleic acid having density corresponding to the reference of determination and amplification of a target nucleic acid having density at which metastasis of cancer to a lymph node is weakly positive.

FIG. 6 is a graph showing the influence of amplification inhibition of an amplification inhibitor that inhibits amplification by PCR of a target nucleic acid and an internal standard nucleic acid.

### DETAILED DESCRIPTION OF THE PREFERRED EMBODIMENT

A genetic testing method of an embodiment of the invention specifically amplifies a DNA complementary to a target nucleic acid existing in a biologic sample, measures the amplified DNA, and generates diagnosis support information on the basis of a measurement result. In the genetic testing method, a DNA complementary to an internal standard nucleic acid is specifically amplified and measured in the presence of a biologic sample component, the result of measurement of the target nucleic acid is compared with a predetermined reference value, and the result of comparison is derived as diagnosis support information. On the basis of a result of measurement of the internal standard nucleic acid, the influence of a nucleic acid amplification inhibitor contained in the biologic sample is determined, or the result of measurement of the target nucleic acid as the base of diagnosis support information is corrected.

In this specification, “amplification of a target nucleic acid” shall include the meaning of “amplification of a DNA complementary to a targeted nucleic acid” for the descriptive purpose.

Additionally, “amplification of an internal standard nucleic acid” shall include the meaning of “amplification of a DNA complementary to an internal standard acid” for the descriptive purpose.

In the genetic testing method of the embodiment, a target nucleic acid is a nucleic acid as an object of nucleic acid amplification measurement, and is DNA, RNA, or the like included in a biologic sample. Concretely, the target nucleic acid is a nucleic acid of a protein which is expressed in a disease or cancer. Target nucleic acids for diagnosing cancer include a nucleic acid of a tumor marker (cancer marker) which does not substantially exist in a normal cell but is specifically expressed in a cancer cell, and a nucleic acid of a protein such as cytokeratin 19 which is expressed to some extent also in a normal cell but is expressed more in a cancer cell. In the case of using, as a target nucleic acid, a nucleic acid of a protein which is expressed to some extent also in a normal cell but is expressed more in a cancer cell, in addition to simple detection of the existence of the target nucleic acid, a reference value proper to the expression amount of the nucleic acid is set and, when the expression amount exceeds the reference value, the existence of a cancer cell is detected.

Examples of the biologic sample are a tissue such as a lymph node extracted from human or animal, whole blood, blood plasma, blood serum, urine, saliva, body fluid, secrete, and the like. Further, a cultured tissue and a cultured cell obtained by cultivating a tissue and a cell extracted from human or animal can be also mentioned.

A nucleic acid containing sample to be provided for nucleic amplification is prepared from a biologic sample. The nucleic acid containing sample is a solution containing a nucleic acid component included in the biologic sample, the nucleic acid component may be purified, or may not be purified. Since it requires efforts and time to purify a nucleic acid component, it is preferable to prepare the nucleic acid containing sample without purifying the nucleic acid component from the viewpoint of prompt preparation of the nucleic acid containing sample. For example, in the case where a biologic sample is a solid tissue such as a lymph node, it is preferable to use, as a nucleic acid containing sample to be subjected to nucleic acid amplification, a solution obtained by homogenizing lymph nodes into a medium by using a breaking tool such as a homogenizer or blender, or supernatant of the solution.

As a medium used for preparing the nucleic acid containing sample, water, water-soluble organic solvent, or the like is used. From the viewpoint of reducing the influence of an amplification inhibitor that inhibits the nucleic acid amplification reaction, it is preferable to use an aqueous solution containing dimethyl sulfoxide as the medium. The concentration of dimethyl sulfoxide in the aqueous solution is, preferably, 1 to 50% (v/v), more preferably, 5 to 30% (v/v) and, further more preferably, 5 to 25% (v/v). The aqueous solution contains, preferably, a surfactant. By using an aqueous solution containing a surfactant, the amount of nucleic acids included in a measurement sample prepared by processing a biologic sample can be increased. As the surfactant, nonionic surfactant, anionic surfactant, cationic surfactant, ampholytic surfactant, and the like can be used, and the nonionic surfactant is preferable. Preferred nonionic surfactants include polyoxyethylene-based nonionic surfactants such as polyoxyethylene alkylether, and polyoxyethylene alkylphenylether.

The nucleic acid containing sample prepared in such a manner is mixed with a reagent for nucleic acid amplification reaction, thereby preparing a measurement sample to be provided for a nucleic acid amplification reaction. Examples of the reagent for nucleic acid amplification reaction include an enzyme reagent containing enzyme such as DNA polymerase and a primer reagent containing a primer for specifically amplifying a target nucleic acid. The measurement sample may be diluted at a predetermined dilute factor. By dilution, the concentration of the amplification inhibitor in the measurement sample decreases, so that the influence of the amplification inhibition of the target nucleic acid can be reduced. However, when the dilute factor is increased, the concentration of the target nucleic acid in the measurement sample also decreases. Consequently, after the relation between required measurement sensitivity of a target nucleic acid and the dilute factor is recognized, the dilute factor has to be determined.

As a nucleic acid amplifying method for amplifying a target nucleic acid in a measurement sample, a known nucleic acid amplifying method can be applied. Examples of the nucleic acid amplifying method are PCR, RT-PCR (Reverse Transcription-Polymerase Chain Reaction), LAMP, RT-LAMP (Reverse Transcription-loop mediated isothermal amplification of DNA), TMA, NASBA (Nucleic Acid Sequence-Based Amplification), 3SR, SDA (Standard Displacement Amplification), and ICAN (Isothermal and Chimeric primer-initiated Amplification of Nucleic acids). Further, a signal amplifying method as a kind of the nucleic acid amplifying method, such as RCA (Rolling Circle Amplification), INVADER, CPT (Cycling Probe Technology), and PALSAR (Probe Alternation Link Self-Assembly Reaction) can be also mentioned. In the signal amplifying method, the target nucleic acid itself is not amplified but a specific nucleotide sequence complementary to the target nucleic acid is amplified. In the case of using mRNA as a target nucleic acid in the RT-PCR or RT-LAMP, mRNA as a target nucleic acid itself is not amplified but cDNA is amplified by using mRNA as a template. Preferred nucleic acid amplifying methods are PCR, RT-PCR, LAMP, and RT-LAMP. In particular, the LAMP and RT-LAMP are more preferable from the viewpoint of swiftness of nucleic acid amplification.

A method of measuring an amplified nucleic acid is not particularly limited. An amplified nucleic acid can be measured by known methods. For example, agarose gel electrophoresis, a real-time fluorescence measuring method for measuring fluorescence by using a probe with a fluorescent label, a turbidity measuring method of measuring turbid (turbidity) due to by-product (magnesium pyrophosphate) occurring at the time of DNA synthesis, a method of determining a base sequence by recognition of a cut pattern due to enzyme as necessary and direct sequence analysis, and many other methods can be used. In the case where the number of nonspecific amplification bands is large and it is difficult to determine a specific band, a specific band can be recognized by, for example, the southern blotting method using a probe in a target amplification range. In particular, it is preferable to promptly measure time since the nucleic acid amplifying reaction rapidly progresses until the number of copies of a nucleic acid rapidly increases (amplification rise time) in a real time manner from the viewpoint of promptly measuring nucleic acid amplification. For example, in the turbidity measuring method, it is preferable to measure time from start of amplification of a nucleic acid until detected turbidity reaches a predetermined value. In the real-time fluorescence measuring method, it is preferable to measure time or the number of cycles since start of amplification of a nucleic acid until fluorescence intensity detected reaches a predetermined value.

An internal standard nucleic acid used for the genetic testing method of the embodiment is a nucleic acid which can be specifically amplified even in the presence of a biologic sample component. Such an internal standard nucleic acid can be specifically amplified without accompanying amplification of various nucleic acids included in the biologic sample at the time of an amplification reaction. That is, the internal standard nucleic acid used in the genetic testing method is a nucleic acid (gene) which does not accompany amplification (nonspecific amplification) of a nucleic acid (gene) included in the biologic sample when the nucleic acid is amplified. As such an internal standard nucleic acid, it is preferable to use a nucleic acid which does never exist in a target biologic sample. By using such a nucleic acid as the internal standard nucleic acid, it becomes easy to select a probe which amplifies a sequence peculiar to the internal standard nucleic acid but does not amplify sequences of various nucleic acids included in a biologic sample. Preferably, the origin of the internal standard nucleic acid is different from that of a nucleic acid existing in a biologic sample. From such a viewpoint, it is preferable to use, as an internal standard nucleic acid for a biologic sample of human, a nucleic acid which does not belong to human. For example, a gene of a protein related to photosynthesis of plant can be employed. Examples of such a gene are ribulosebisphosphate carboxylase small chain 1A gene and mRNA of the gene. A preferable gene is a gene derived from an Arabidopsis plant. An internal standard nucleic acid obtained by mutating a part of a base sequence of a target nucleic acid described in Japanese Patent Laid-Open No. 2004-203 and the like can be also used. In the case of using such an internal standard nucleic acid, however, the selection of conditions for performing specific amplification even in the presence of a biologic sample component is narrowed.

With respect to the internal standard nucleic acid, the relation with specific amplification of a target nucleic acid in a biologic sample is known, and the amount (concentration or the number of copies) of the internal standard nucleic acid corresponding to a predetermined amount (predetermined concentration or a predetermined number of copies) of the target nucleic acid is known. The amplification efficiency of a preferred internal standard nucleic acid is almost equal to that of a target nucleic acid.

It is necessary to use an internal standard nucleic acid whose amplification is inhibited by an amplification inhibitor included in the biologic sample. Preferably, the degree of inhibition of amplification by an amplification inhibitor is similar or close to that for a target nucleic acid in a biologic sample. On the other hand, in the case of using an internal standard nucleic acid in which the degree of the influence of amplification inhibition is different from that in the target nucleic acid, it is sufficient to preliminarily obtain the difference between the influences of inhibition by the amplification inhibitor and correct it. That is, it is preferable that the relation between the degree of inhibition of amplification of the internal standard nucleic acid by the amplification inhibitor and the degree of inhibition of amplification of the target nucleic acid by the amplification inhibitor be known.

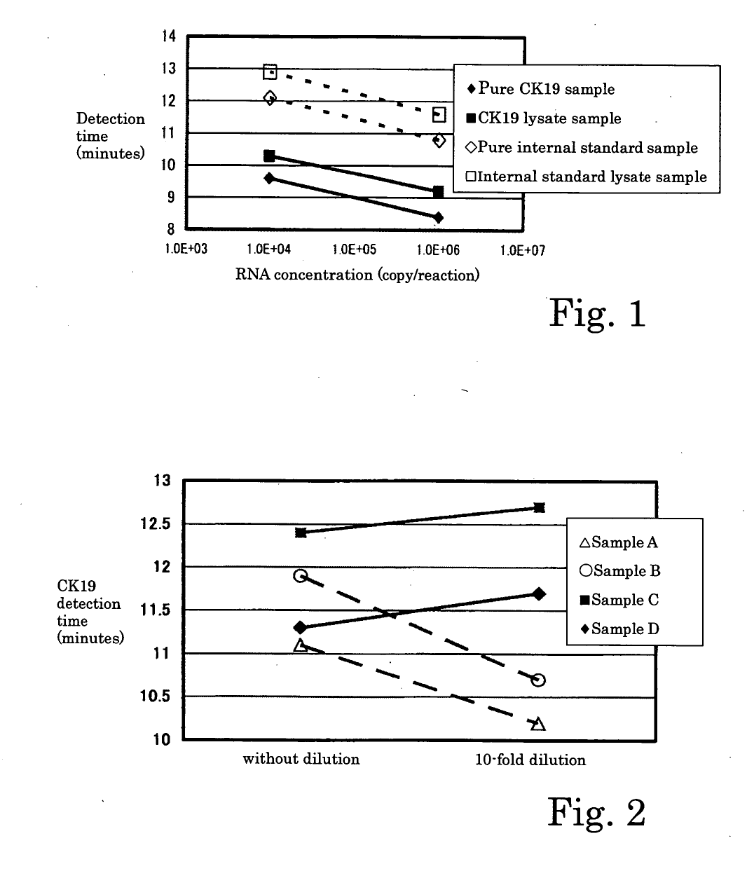

An example of an internal standard nucleic acid in which the degree of inhibition by an amplification inhibitor is similar to that in a target nucleic acid in a biologic sample will be described with reference to FIG. 1. In FIG. 1, “♦ (pure CK19 sample)” is a plot showing the relation between time (detection time) required for turbidity to become 0.1 and mRNA concentration when nucleic acid amplification is performed by RT-LAMP on a measurement sample which does not contain an amplification inhibitor but contains 1×10⁴ copies/reaction of mRNA of cytokeratin 19 (CK19) as a target nucleic acid and a measurement sample containing 1×10⁶ copies/reaction of mRNA of CK19, and opacity of magnesium pyrophosphate generated as by-product of the nucleic acid amplification is measured as turbidity. “▪ (CK19 lysate sample) is a plot showing the relation of time required for turbidity to become 0.1 and RNA concentration obtained in a manner similar to the above except that the measurement sample contains an amplification inhibitor. In other words, the graph of “♦” shows the result of performing amplification of Cytokeratin 19 in the absence of an amplification inhibitor, and the graph of “▪” shows the result of performing amplification of Cytokeratin 19 in the presence of an amplification inhibitor. From comparison of the lines, it is understood that amplification of Cytokeratin 19 is inhibited by the existence of an amplification inhibitor, and detection time is increased.

In FIG. 1, “⋄ (pure internal standard sample) is a plot showing the relation between time required for turbidity to become 0.1 and mRNA concentration when nucleic acid amplification is performed by RT-LAMP on a measurement sample which does not contain an amplification inhibitor but contains 1×10⁴ copies/reaction of mRNA of an internal standard nucleic acid (Arabidopsis) and a measurement sample containing 1×10⁶ copies/reaction of mRNA of the internal standard nucleic acid, and opacity of magnesium pyrophosphate generated as by-product of the nucleic acid amplification is measured as turbidity. “□ (internal standard lysate sample)” is a plot showing the relation of time required for turbidity to become 0.1 and RNA concentration obtained in a manner similar to the above except that nucleic acid amplification is performed under the condition that the measurement sample contains an amplification inhibitor. From comparison of the lines of “⋄” and “□”, it is understood that amplification of the internal standard nucleic acid is inhibited by the existence of an amplification inhibitor, and detection time is increased. It is also understood that delay in detection time of Cytokeratin 19 and that of the internal standard nucleic acid are similar to each other, and the amplification efficiency of Cytokeratin 19 and that of the internal standard nucleic acid are almost the same.

Such an internal standard nucleic acid can be selected by the following method. First, various primers are prepared as primers for amplifying mRNA of a gene to be an internal standard nucleic acid. It is sufficient to measure the nucleic acid amplification of the gene by using each of the various primers and select, as an internal standard nucleic acid, a gene capable of selecting a primer in which the degree of delay in the nucleic acid amplification due to inhibition is similar to that in the target nucleic acid.

A first genetic testing method of the present invention specifically amplifies a target nucleic acid existing in a biologic sample, measures the amplified target nucleic acid and, on the basis of a measurement result, generates diagnosis support information, and includes: a step of specifically amplifying an internal standard nucleic acid of a known amount corresponding to a predetermined amount of the target nucleic acid, which can be specifically amplified in the presence of the biologic sample component, in the absence of the biologic sample component and, on the basis of the amplification, obtaining a first measurement result; a step of specifically amplifying the known amount of the internal standard nucleic acid in the presence of the biologic sample component and, on the basis of the amplification, obtaining a second measurement result; a step of specifically amplifying the target nucleic acid in the presence of the biologic sample component and, on the basis of the amplification, obtaining a third measurement result; a step of obtaining diagnosis support information on the basis of the third measurement result and a predetermined reference value; and a step of determining the influence on the diagnosis support information of an amplification inhibitor in the biologic sample component.

In the first gene testing method, first, an internal standard nucleic acid containing reference sample which contains a predetermined amount (a predetermined number of copies) of an internal standard nucleic acid is prepared. Since the internal standard nucleic acid containing reference sample does not contain a biologic sample component, it does not contain an amplification inhibitor. The internal standard nucleic acid containing reference sample is mixed with an enzyme reagent containing enzyme such as DNA polymerase, a primer reagent containing a primer for specifically amplifying the internal standard nucleic acid, or the like to prepare a measurement sample. The measurement sample is provided for a nucleic acid amplification reaction. By a predetermined nucleic acid amplifying method, the internal standard nucleic acid is specifically amplified. The amplification is measured to obtain a first measurement result.

Next, an internal standard nucleic acid containing sample obtained by adding a predetermined amount (a predetermined number of copies) of an internal standard nucleic acid to a nucleic acid containing sample prepared from a biologic sample is prepared. The internal standard nucleic acid containing sample, an enzyme reagent containing enzyme such as DNA polymerase, a primer reagent containing a primer for specifically amplifying the internal standard nucleic acid, and the like are mixed to prepare a measurement sample. The measurement sample is provided for a nucleic acid amplification reaction. Specific amplification of the internal standard nucleic acid performed in the presence of the biologic sample component is measured, thereby obtaining a second measurement result.

A nucleic acid containing sample prepared from a biologic sample, an enzyme reagent containing enzyme such as DNA polymerase, a primer reagent containing a primer for specifically amplifying a target nucleic acid, and the like are mixed to prepare a measurement sample, and the measurement sample is provided for a nucleic acid amplification reaction. Specific amplification of the target nucleic acid contained in the biologic sample is measured, thereby obtaining a third measurement result.

The obtained third measurement result is compared with a predetermined reference value, thereby generating diagnosis support information. In this case, the reference value corresponds to a measurement value obtained at the time of amplifying a reference amount of target nucleic acids. The reference amount of the target nucleic acids (reference target nucleic acid amount) corresponds to, for example, in the case where a biologic sample is a lymph node and metastasis of cancer to lymph nodes is diagnosed, an amount of target nucleic acids contained in a cancer tissue having a size as a threshold at which metastasis of cancer to lymph nodes is determined. That is, the reference amount of the target nucleic acid is a target nucleic acid amount as a threshold for determining whether metastasis of cancer to lymph nodes is positive or not. Therefore, the diagnosis support information, as support information used by a doctor or the like to diagnose a disease, metastasis of cancer, or the like, is a result of determination whether a measured target nucleic acid amount is larger than the target nucleic acid amount as a reference for determining disease, the presence of cancer, or the metastasis of cancer. It is preferable to provide, as diagnosis support information, a result of determination of whether metastasis is positive or negative on the basis of the result of comparison or a result of determination of whether it is normal or abnormal. The reference value corresponds to a result of measurement of specific amplification of a target nucleic acid on a positive control containing a reference amount of the target nucleic acid. It is also possible to perform specific amplification of the target nucleic acid on a plurality of positive controls having different contents of the target nucleic acids, generate a standard curve from the result of measurement of the amplification, and obtain the reference value from the standard curve.

It is also possible to compare the third measurement result with first and second reference values and, on the basis of the result of comparison, obtain diagnosis support information. In this case, preferably, for example, the first reference value corresponds to a target nucleic acid amount as a threshold for determining whether cancer metastasis is positive or weakly positive, and the second reference value corresponds to a target nucleic acid amount as a threshold for determining whether cancer metastasis is weakly positive or negative. Preferably, the determination result is displayed as positive (++), weakly positive (+), or negative (−). It is also possible to display the result as positive (+), gray (+), or negative (−).

In the first gene testing method, the influence on diagnosis of an amplification inhibitor in a biologic sample is determined on the basis of the first and second measurement results. In this case, the first measurement result is a measurement result of amplification of an internal standard nucleic acid performed in the absence of an amplification inhibitor, and the second measurement result is a measurement result of amplification of an internal standard nucleic acid performed in the presence of an amplification inhibitor. Consequently, by comparing the first and second measurement results, the degree of inhibition of nucleic acid amplification of the internal standard nucleic acid by the amplification inhibitor can be obtained. In this case, the relation between the degree of inhibition of nucleic acid amplification of the target nucleic acid by the amplification inhibitor and the degree of inhibition of the nucleic acid amplification of the internal standard nucleic acid by the amplification inhibitor is known. Consequently, by comparing the first and second measurement results, the degree of nucleic acid amplification inhibition by the amplification inhibitor can be obtained. Concretely, in the case of using an internal standard nucleic acid on which the influence of inhibition is similar to that of a target nucleic acid, by comparing the difference between first and second measurement results with a predetermined threshold, whether or not the influence of an amplification inhibitor is large on the third measurement result or diagnosis support information based on the third measurement result can be determined. When it is determined that the influence of the amplification inhibitor is large, preferably, a warning about the reliability of diagnosis support information is issued. In the case where it is determined that the influence of an amplification inhibitor is large, in order to reduce the influence of the amplification inhibitor, it is also possible to dilute a sample provided for amplification of a target nucleic acid at a predetermined factor, amplify the target nucleic acid in the diluted sample, measure the nucleic acid amplification, and thereby obtain the third measurement result again. It is also possible to correct the third measurement result or the reference value on the basis of the difference between the first and second measurement results and, on the basis of the correction result, obtain diagnosis support information.

A second genetic testing method of the present invention specifically amplifies a target nucleic acid existing in a biologic sample, measures the amplified target nucleic acid and, on the basis of a measurement result, generates diagnosis support information, and includes: a step of specifically amplifying an internal standard nucleic acid of a known amount corresponding to a predetermined amount of the target nucleic acid, which can be specifically amplified in the presence of the biologic sample component, in the absence of the biologic sample component and, on the basis of the amplification, obtaining a first measurement result; a step of specifically amplifying the known amount of the internal standard nucleic acid in the presence of the biologic sample component and, on the basis of the amplification, obtaining a second measurement result; a step of specifically amplifying the target nucleic acid in the presence of the biologic sample component and, on the basis of the amplification, obtaining a third measurement result; a step of correcting either the third measurement result or a reference value corresponding to a reference target nucleic acid amount on the basis of first and second measurement results; and a step of obtaining diagnosis support information on the basis of the corrected one of the third measurement result and the reference value, and the other. Since the steps for obtaining the first to third measurement results are similar to those of the above-described first genetic testing method, description will not be repeated.

In the second gene testing method, after the first to third measurement results are obtained, on the basis of the first and second measurement results, one of the third measurement result and a reference value corresponding to a reference target nucleic acid amount is corrected. In this case, it is also possible to obtain a correction value on the basis of the first and second measurement results and make a correction on the basis of the correction value. The correction value is a correction value for correcting the influence on diagnosis of an amplification inhibitor in a biologic sample. As described above, the first measurement result is a measurement result of amplification of an internal standard nucleic acid in the absence of an amplification inhibitor, and the second measurement result is a measurement result of amplification of the internal standard nucleic acid in the presence of the amplification inhibitor. Consequently, by comparing the first and second measurement results, the degree of nucleic acid amplification inhibition on the target nucleic acid by the amplification inhibitor can be obtained. To be concrete, in the case of using an internal standard nucleic acid on which the influence of inhibition is similar to that on the target nucleic acid, the difference between the first and second measurement results can be used as a correction value.

Next, on the basis of the reference value and the corrected third measurement result, or on the basis of the third measurement result and a corrected reference value, diagnosis support information is obtained. To be concrete, by comparing a result of correcting the third measurement result on the basis of the correction value with a reference value, diagnosis support information is obtained. In the case where the correction value is the difference between the first and second measurement results, the difference between the third measurement result and the correction value can be obtained as the measurement result of amplification of the target nucleic acid. In the measurement result of amplification, the influence of the amplification inhibitor is corrected. Therefore, by comparing the result with a reference value, whether the amount is larger than the reference target nucleic acid amount or not can be determined accurately. That is, as diagnosis support information used by a doctor or the like to diagnosis disease, metastasis of cancer, or the like, a result of determination of whether a measured target nucleic acid amount is larger than a target nucleic acid amount as a reference (reference target nucleic acid amount) of determining the presence of disease or cancer or metastasis of cancer, in which the influence of the amplification inhibitor is corrected, is accurately provided. As diagnosis support information, a result of determination of whether metastasis of cancer is positive or not or a result of determination of whether it is normal or abnormal may be provided. The reference value corresponds to a result of measurement of specific amplification of a target nucleic acid on a positive control containing a reference amount of the target nucleic acid. It is also possible to perform specific amplification of the target nucleic acid on a plurality of positive controls having different contents of the target nucleic acids, generate a standard curve from the result of measurement of the amplification, and obtain the reference value from the standard curve.

Alternately, the third measurement result may be compared with first and second reference values, on the basis of the result of comparison, to obtain diagnosis support information. In this case, preferably, for example, the first reference value corresponds to a target nucleic acid amount as a threshold for determining whether cancer metastasis is positive or weakly positive, and the second reference value corresponds to a target nucleic acid amount as a threshold for determining whether cancer metastasis is weakly positive or negative. Preferably, the determination result is displayed as positive (++), weakly positive (+), or negative (−). It is also possible to display the result as positive (+), gray (±), or negative (−).

Although the third measurement result is corrected with a correction value, the reference value may be also corrected with a correction value. To be concrete, diagnosis support information is obtained on the basis of a correction reference value obtained by correcting the reference value with a correction value, and the third measurement result. In the case of using the first and second reference values, it is sufficient to correct the first and second reference values on the basis of the correction value to obtain first and second corrected reference values and, on the basis of the first and second correction reference values and a third measurement result, obtain diagnosis support information. Also in the first genetic testing method, the third measurement result or the reference value may be corrected by using a correction value obtained from the first and second measurement results.

A third genetic testing method of the invention specifically amplifies a target nucleic acid existing in a biologic sample, measures the amplified target nucleic acid and, on the basis of a measurement result, generates diagnosis support information, and includes: a step of specifically amplifying an internal standard nucleic acid of a predetermined amount corresponding to a reference target nucleic acid amount, which can be specifically amplified in the presence of the biologic sample component, in the presence of the biologic sample component and, on the basis of the amplification, obtaining a first measurement result; a step of specifically amplifying the target nucleic acid in the presence of the biologic sample component and, on the basis of the amplification, obtaining a second measurement result; and a step of comparing the first and second measurement results to obtain diagnosis support information.

In the third genetic testing method, first, a predetermined amount of an internal standard nucleic acid corresponding to a reference target nucleic acid amount is specifically amplified in the presence of the biologic sample component and, on the basis of the amplification, the first measurement result is obtained. The first measurement result is an amplification measurement result of the internal standard nucleic acid measured under the condition in which the influence of the amplification inhibitor in the biologic sample component is exerted. The amount of the internal standard nucleic acid provided for measurement is an amount corresponding to the reference target nucleic acid amount. As described above, the reference target nucleic acid amount is a target nucleic acid amount as a threshold for determining whether disease, cancer, or metastasis of cancer is positive or not. Therefore, the first measurement result is a measurement value corresponding to a reference target nucleic acid amount in which the influence of the amplification inhibitor is reflected.

Next, the target nucleic acid is specifically amplified and, on the basis of the amplification, a second measurement result is obtained. The second measurement result is a measurement result of amplification of a target nucleic acid measured by a method similar to that of the third measurement result in the first gene testing method and is a measurement value in which the influence of an amplification inhibitor is reflected. Therefore, by comparing the first measurement result corresponding to the reference target nucleic acid amount with the second measurement result, a result of determination of whether the target nucleic acid amount contained in the biologic sample is larger than the reference target nucleic acid amount or not can be obtained as diagnosis support information. Alternately, as diagnosis support information, the first and second measurement results may be compared with each other to determine whether disease, cancer or metastasis is positive or negative.

Alternately, it is also possible to measure a result of amplification of a first predetermined amount of an internal standard nucleic acid corresponding to the first reference target nucleic acid amount and that of a second predetermined amount of an internal standard nucleic acid corresponding to the second reference target nucleic acid amount, and compare the measurement result with the second measurement result to determine a target nucleic acid amount on the basis of the two reference values, thereby obtaining diagnosis support information.

### EXAMPLE 1

By using mRNA of human cytokeratin 19 (hereinbelow, described as CK19) and mRNA of RBCS-1A as templates, cDNA was amplified by RT-LAMP in the presence of an amplification inhibitor and in the absence of an amplification inhibitor, and an analysis was conducted to see how the amplification inhibitor exerts an influence on nucleic acid amplification. mRNA of CK19 is a target nucleic acid for determining metastasis of breast cancer to lymph nodes. The sequence (sequence number 1) of cDNA amplified by using mRNA of CK19 as a template and the sequence (sequence number 2) of cDNA amplified by using mRNA of RBCS-1A as a template are shown.

Preparation of Reaction Mixture
13.97 μl of a reaction mixture was prepared by
mixing the following components.

| | |
|---|---|
| 750 mM tris buffer (pH8.0) | 1.00 μl |
| 10X Thermopol buffer (from New England Bio Labs Ltd.) | 2.50 μl |
| 10 mM dNTPs | 2.00 μl |
| 100 mM MgSO₄ | 0.75 μl |
| 100 mM Dithiothreitol | 1.25 μl |
| 2% Tergitol (Sigma-Aldrich Japan K.K.) | 2.50 μl |
| H₂O | 3.97 μl |

Preparation of Enzyme Reagent
3.04 μl of an enzyme reagent was prepared by mixing
the following components.

| | |
|---|---|
| 10 U/μl AMV reverse transcriptase (Promega Co.) | 0.14 μl |
| 8 U/μl Bst DNA polymerase (from New England Bio Labs | 2.27 μl |
| Ltd.) | |
| RNase amplification inhibitor (Promega Co.) | 0.63 μl |

Preparation of Primer Reagent 1
6.00 μ of a primer reagent 1 was prepared by mixing
the following components.

| | |
|---|---|
| 80 pmol/μl forward inner primer | 1.00 μl |
| (sequence number 3: | |
| ggagttctcaatggtggcaccaactactacacgaccatcca) | |
| 80 pmol/μl reverse inner primer | 1.00 μl |
| (sequence number 4: | |
| gtcctgcagatcgacaacgcctccgtctcaaacttggttcg) | |
| 5 pmol/μl forward outer primer | 1.00 μl |
| (sequence number 5: tggtaccagaagcagggg) | |
| 5 pmol/μl reverse outer primer | 1.00 μl |
| (sequence number 6: gttgatgtcggcctccacg) | |
| 60 pmol/μl forward loop primer | 1.00 μl |
| (sequence number 7: agaatcttgtcccgcagg) | |
| 60 pmol/μl reverse loop primer | 1.00 μl |
| (sequence number 8: cgtctggctgcagatga) | |

Preparation of Primer Reagent 2
6.00 μ of a primer reagent 2 was prepared by mixing
the following components.

| | |
|---|---|
| 80 pmol/μl forward inner primer | 1.00 μl |
| (sequence number 9: | |
| accgaacaagggaagcttccactgagcacggtaactcaccc) | |
| 80 pmol/μl reverse inner primer | 1.00 μl |
| (sequence number 10: | |
| accgactccgctcaagtgttg-tcctaatgaaggcattgggg) | |
| 5 pmol/μl forward outer primer | 1.00 μl |
| (sequence number 11: tggagcacggatttgtgtac) | |
| 5 pmol/μl reverse outer primer | 1.00 μl |
| (sequence number 12: cactggacttggcgggtg) | |
| 60 pmol/μl forward loop primer | 1.00 μl |
| (sequence number 13: ccagtaccgtccatcatag) | |
| 60 pmol/μl reverse loop primer | 1.00 μl |
| (sequence number 14: gaagtggaagagtgcaagaa) | |

Preparation of RT-LAMP Reaction Mixtures A and B

An RT-LAMP reaction mixture A made by the reaction mixture, the enzyme reagent, and the primer reagent 1 was prepared. The RT-LAMP reaction mixture A is a reaction mixture for amplifying cDNA by the RT-LAMP using mRNA of CK19 as a template.

An RT-LAMP reaction mixture B made by the reaction mixture, the enzyme reagent, and the primer reagent 2 was prepared. The RT-LAMP reaction mixture B is a reaction mixture for amplifying cDNA by the RT-LAMP using mRNA of RBCS-1A as a template.

Preparation of Solubilization Reagent

A solubilization reagent containing the following components was prepared.

200 mM (pH3.0) Glycin-HCl buffer

20% (v/v) dimethyl sulfoxide

5% nonionic surfactant Brij35 (from Sigsa)

0.05% antifoaming agent KS-538 (from Shin-Etsu Chemical Co., Ltd.)

Preparation of Model Sample X

100 μl of a solubilization reagent was added to lymph nodes of human with negative cancer metastasis removed during a breast cancer operation, and the resultant was homogenized at 12,000 rpm by a metal blender. The resultant is dispensed by 30 ml and four model samples X were generated. In the model sample X, a substance which inhibits nucleic acid amplification is contained but mRNA of CK19 is hardly expressed.

Preparation of Measurement Sample “a”

A solution obtained by adding 1×10⁶ (copies/reaction) of mRNA of CK19 to 2μl of a solubilization reagent was prepared. The solution was added to 23 μl of the RT-LAMP reaction mixture A prepared in Example 1, thereby preparing a measurement sample “a”.

Preparation of Measurement Sample “b”

A solution obtained by adding 1×10⁴ (copies/reaction) of mRNA of CK19 to 2 μl of a solubilization reagent was prepared. The solution was added to 23 μl of the RT-LAMP reaction mixture A, thereby preparing a measurement sample “b”.

Preparation of Measurement Sample “c”

A solution obtained by adding 1×10⁶ (copies/reaction) of mRNA of RBCS-1A to 2 μl of a solubilization reagent was prepared. The solution was added to 23 μl of the RT-LAMP reaction mixture B, thereby preparing a measurement sample “c”.

Preparation of Measurement Sample “d”

A solution obtained by adding 1×10⁴ (copies/reaction) of mRNA of RBCS-1A to 2 μl of a solubilization reagent was prepared. The solution was added to 23 μl of the RT-LAMP reaction mixture B, thereby preparing a measurement sample “d”.

Preparation of Measurement Sample “e”

A solution obtained by adding 1×10⁶ (copies/reaction) of mRNA of CK19 to the model sample X was prepared. 2 μl of the solution was added to 23 μl of the RT-LAMP reaction mixture A, thereby preparing a measurement sample “e”.

Preparation of Measurement Sample “f”

A solution obtained by adding 1×10⁴ (copies/reaction) of mRNA of CK19 to the model sample X was prepared. 2 μl of the solution was added to 23 μl of the RT-LAMP reaction mixture A, thereby preparing a measurement sample “f”.

Preparation of Measurement Sample “g”

A solution obtained by adding 1×10⁶ (copies/reaction) of mRNA of RBCS-1A to the model sample X was prepared. 2 μl of the solution was added to 23 μl of the RT-LAMP reaction mixture B, thereby preparing a measurement sample “g”.

Preparation of Measurement Sample “h”

A solution obtained by adding 1×10⁴ (copies/reaction) of mRNA of RBCS-1A to the model sample X was prepared. 2 μl of the solution was added to 23 μl of the RT-LAMP reaction mixture B, thereby preparing a measurement sample “h”.

Since the measurement samples “a” to “d” do not contain the model sample X, no amplification inhibitor is contained. Since the model sample X is added to the measurement samples “e” to “h”, the amplification inhibitor is contained. A schematic composition of each of the measurement samples is shown in Table 1.

TABLE 1

| | | | Nucleic | |
|---|---|---|---|---|
| | | | acid | |
| | | | addition | |
| | | Nucleic | amount | RT-LAMP |
| | Model | acid | (copies/ | reaction |
| | sample | (mRNA) | reaction) | mixture |
| Measurement | not | CK19 | 1 × 10⁶ | A (for CK19 |
| sample a | contained | | | measurement) |
| Measurement | not | CK19 | 1 × 10⁴ | A (for CK19 |
| sample b | contained | | | measurement) |
| Measurement | not | RBCS-1A | 1 × 10⁶ | B (for |
| sample c | contained | | | RBCS-1A |
| | | | | measurement) |
| Measurement | not | RBCS-1A | 1 × 10⁴ | B (for |
| sample d | contained | | | RBCS-1A |
| | | | | measurement) |
| Measurement | contained | CK-19 | 1 × 10⁶ | A (for CK19 |
| sample e | | | | measurement) |
| Measurement | contained | CK-19 | 1 × 10⁴ | A (for CK19 |
| sample f | | | | measurement) |
| Measurement | contained | RBCS-1A | 1 × 10⁶ | B (for |
| sample g | | | | RBCS-1A |
| | | | | measurement) |
| Measurement | contained | RBCS-1A | 1 × 10⁴ | B (for |
| sample h | | | | RBCS-1A |
| | | | | measurement) |

Nucleic Acid Amplification by RT-LAMP and Measurement of the Amplification

With LA-200 from TERAMECS Co., Ltd., opacity of insoluble magnesium pyrophosphate generated as by-product simultaneously with nucleic acid amplification was measured in a real-time manner.

Time (detection time) since cDNA corresponding to mRNA contained in each of the measurement samples is amplified by the RT-LAMP until the turbidity reaches 0.1 was measured. FIG. 1 shows the measurement results.

In FIG. 1, ♦ (pure CK19 sample) shows measurement results of the measurement samples “a” and “b” (measurement results of nucleic acid amplification of CK19 in the absence of an amplification inhibitor).

⋄ (pure internal standard sample) indicates measurement results of the measurement samples “c” and “d” (measurement results of nucleic acid amplification of RBCS-1A in the absence of an amplification inhibitor).

▪ (CK19 lysate sample) shows measurement results of the measurement samples “e” and “f” (measurement results of nucleic acid amplification of CK19 in the presence of an amplification inhibitor).

□ (internal standard lysate sample) shows measurement results of the measurement samples “g” and “h” (measurement results of nucleic acid amplification of RBCS-1A in the presence of an amplification inhibitor).

In FIG. 1, by comparison between ♦ and ▪, delay in detection time due to the presence of the amplification inhibitor in the nucleic acid amplification of CK19 can be obtained.

By comparison between ⋄ and □, delay in detection time due to the presence of the amplification inhibitor in the nucleic acid amplification of RBCS-1A can be obtained.

From FIG. 1, it is understood that the delay in the detection time in the nucleic acid amplification of CK19 and that in the detection time in the nucleic acid amplification of RBCS-1A are almost the same, and the influence of nucleic acid amplification inhibition by the amplification inhibitor in CK19 and that in RBCS-1A are almost the same.

From the results of Example 1, it was found that by using, as an internal standard, the nucleic acid of RBCS-1A whose concentration is known, whether the amplification inhibitor exerts an influence on the nucleic acid amplification of CK19 or not can be detected. Further, it was also found that, in the case where the amplification inhibitor exerts an influence on the nucleic acid amplification, the degree of the influence (delay in detection time) on the nucleic acid amplification of CK19 can be obtained on the basis of the degree of the influence (delay in detection time) on the nucleic acid amplification of the RBCS-1A.

### EXAMPLE 2

Preparation of Nucleic Acid Containing Samples A to D

4 ml of a solubilization reagent was added to lymph nodes A of human with positive cancer metastasis isolated by a breast cancer operation, and the resultant was homogenized at 12,000 rpm by a metal blender, thereby preparing a nucleic acid containing sample A. Similarly, nucleic acid containing samples B to D were prepared with respect to lymph nodes B to D of human with positive cancer metastasis isolated by a breast cancer operation.

Preparation of Measurement Samples a1 to d1

2 μl of a solution obtained by adding 1×10⁶ (copies/reaction) of mRNA of RBCS-1A as an internal standard nucleic acid to the nucleic acid containing sample A and mixing the resultant for 30 seconds by a vortex mixer was added to 23 μl of an RT-LAMP reaction mixture A (RT-LAMP reaction mixture for measuring CK19), thereby preparing a measurement sample a1.

In a manner similar to the preparation of the measurement sample a1 except that the nucleic acid containing samples B to D were used in place of the nucleic acid containing sample A, measurement samples b1 to d1 were prepare d.

Preparation of Measurement Samples a2 to d2

In a manner similar to the preparation of the measurement samples a1 to d1 except that an RT-LAMP reaction mixture B (RT-LAMP reaction mixture for measuring RBCS-1A) was used in place of the RT-LAMP reaction mixture A, measurement samples a2 to d2 were prepared.

Preparation of Measurement Samples a3 to d3 (10-Fold Dilution)

2 μl of a solution obtained by diluting the nucleic acid containing sample A by 10 times with a solubilization reagent, adding 1×10⁶ (copies/reaction) of mRNA of RBCS-1A to the resultant, and mixing the resultant for 30 seconds by a vortex mixer was added to 23 μl of the RT-LAMP reaction mixture A, thereby preparing the measurement sample a3.

In a manner similar to the preparation of the measurement sample a2 except that nucleic acid containing samples B to D were used in place of the nucleic acid containing sample A, measurement samples b3 to d3 were prepared.

Preparation of Measurement Samples a4 to d4 (10-Fold Dilution)

In a manner similar to the preparation of the measurement samples a1 to d1 except that the RT-LAMP reaction mixture B was used in place of the RT-LAMP reaction mixture A, measurement samples a4 to d4 were prepared.

Preparation of Measurement Sample g2 (10-Fold Dilution)

In a manner similar to the preparation of the measurement sample “g” in Example 1 except that the model sample X diluted by 10 times with a solubilization reagent was used, a measurement sample g2 was prepared. A schematic composition of each of the measurement samples is shown in Table 2.

TABLE 2

| | | Nucleic acid | RT-LAMP |
|---|---|---|---|
| | | containing | reaction |
| | Sample | sample | mixture |
| Measurement | A | A (not diluted) | A (for CK19 |
| sample a1 | | | measurement) |
| Measurement | B | B (not diluted) | A (for CK19 |
| sample b1 | | | measurement) |
| Measurement | C | C (not diluted) | A (for CK19 |
| sample c1 | | | measurement) |
| Measurement | D | D (not diluted) | A (for CK19 |
| sample d1 | | | measurement) |
| Measurement | A | A (not diluted) | B (for RBCS-1A |
| sample a2 | | | measurement) |
| Measurement | B | B (not diluted) | B (for RBCS-1A |
| sample b2 | | | measurement) |
| Measurement | C | C (not diluted) | B (for RBCS-1A |
| sample c2 | | | measurement) |
| Measurement | D | D (not diluted) | B (for RBCS-1A |
| sample d2 | | | measurement) |
| Measurement | A | A (diluted by | A (for CK19 |
| sample a3 | | ten times) | measurement) |
| Measurement | B | B (diluted by | A (for CK19 |
| sample b3 | | ten times) | measurement) |
| Measurement | C | C (diluted by | A (for CK19 |
| sample c3 | | ten times) | measurement) |
| Measurement | D | D (diluted by | A (for CK19 |
| sample d3 | | ten times) | measurement) |
| Measurement | A | A (diluted by | B (for RBCS-1A |
| sample a4 | | ten times) | measurement) |
| Measurement | B | B (diluted by | B (for RBCS-1A |
| sample b4 | | ten times) | measurement) |
| Measurement | C | C (diluted by | B (for RBCS-1A |
| sample c4 | | ten times) | measurement) |
| Measurement | D | D (diluted by | B (for RBCS-1A |
| sample d4 | | ten times) | measurement) |
| Measurement | X | X (not diluted) | B (for RBCS-1A |
| sample g | | | measurement) |
| Measurement | X | X (diluted by | B (for RBCS-1A |
| sample g1 | | ten times) | measurement) |

With LA-200 from TERAMECS Co., Ltd., cDNA corresponding to mRNA contained in each of the measurement samples was amplified by RT-LAMP and time (detection time) required until turbidity of each of the measurement samples reaches 0.1 was measured. Measurement results are shown in FIG. 2 and Table 3.

TABLE 3

| | without dilution | | | |
|---|---|---|---|---|
| | Fluctuations in | | | |
| | detection time of | | | |
| | internal standard | | Detection | |
| | | Detection | time of CK19 | |
| | | time | | Detection |
| | Measurement | difference | Measurement | time |
| | sample | (minute) | sample | (minutes) |
| Sample A | a2-g | 0.9(>0.6) | a1 | 11.1 |
| Sample B | b2-g | 1.6(>0.6) | b1 | 11.9 |
| Sample C | c2-g | 0.3(<0.6) | c1 | 12.4 |
| Sample D | d2-g | 0.2(<0.6) | d1 | 11.3 |
| | 10-fold dilution | | | |
| | Fluctuations in | | | |
| | detection time of | | Detection | |
| | internal standard | | time of CK19 | |
| | | Detection | | Detection |
| | | time | | time |
| | Measurement | difference | Measurement | difference |
| | sample | (minute) | sample | (minutes) |
| Sample A | a4-g1 | −0.3 | a3 | 10.2 |
| Sample B | b4-g1 | 0.2 | b3 | 10.7 |
| Sample C | c4-g1 | −0.1 | c3 | 12.7 |
| Sample D | d4-g1 | −0.1 | d3 | 11.7 |

As shown in Table 3, in the case where the sample A is not diluted, detection time of the internal standard nucleic acid is delayed by 0.9 minute more than detection time of the internal standard nucleic acid in the absence of the amplification inhibitor. The detection time of the sample B is delayed by 1.6 minutes. That is, the fluctuation value (detection time difference) of the internal standard nucleic acid of each of the samples A and B is larger than 0.6 minute as the threshold. As shown in FIG. 2, when the case of measuring detection time of a target gene (mRNA of CK19) in each of the sample A (Δ in the diagram) and the sample B (O in the diagram) without diluting the samples is compared with the case of measuring detection time in a state where the samples are diluted by 10 times, in spite of the fact that the samples are diluted by 10 times, the detection time in the latter case is shorter. That is, it is understood that the samples A and B are samples in which the influence of the amplification inhibitor on the nucleic acid amplification is strong.

On the other hand, the fluctuation value (detection time difference) of the internal standard nucleic acid of the sample C is 0.3 minute, and that of the sample D is 0.2 minute. Each of the fluctuation values is less than 0.6 minute as a threshold. As shown in FIG. 2, detection time of the target gene (mRNA of CK19) in each of the samples C (▪ in the diagram) and the sample D (♦ in the diagram) in the case where the samples are diluted by 10 times is longer only by the amount of dilution.

Therefore, in the case where the detection time of the internal standard nucleic acid added to the sample and detection time in the absence of an amplification inhibitor are compared with each other and the detection time fluctuates by predetermined time or longer, flagging can be made that the measurement value is a value influenced by inhibition. Specifically, when the fluctuation value (detection time difference) of the internal standard nucleic acid is larger than the threshold, the amount of carry-in of the amplification inhibitor to the gene amplification reaction system is reduced by diluting the sample, and measurement can be performed again. Alternately, the sample is coarsely purified or is normally purified so as to obtain a state where there is no inhibition, and measurement can be performed again. By making accurate determination by such a method, in a site of an operation or the like where an extremely strict determination is requested, false positive and the like can be reduced largely. By using the fluctuation value (detection time difference) of the internal standard nucleic acid, detection time of the target gene can be corrected.

### EXAMPLE 3

Preparation of Measurement Samples 3A to 5A and 3B to 5B

1.5×10⁸ (copies/reaction) mRNA of RBCS-1A (corresponding to 2.5×10⁵ (copies/reaction) of mRNA of CK19) and 2.5×10⁵ (copies/reaction) of mRNA of CK19 are added to the model sample X, and the resultant was mixed by a vortex mixer for 30 seconds. 2 μl of the obtained solution was added to 23 μl of the RT-LAMP reaction mixture A, thereby preparing a measurement sample 3A.

A measurement sample 3B was prepared in a manner similar to the measurement sample 3A except that the RT-LAMP reaction mixture A was changed to the RT-LAMP reaction mixture B.

A measurement sample 4A was prepared in a manner similar to the measurement sample 3A except that the addition amount of the mRNA of CK19 was changed to 2.5×10⁶ (copies/reaction).

A measurement sample 4B was prepared in a manner similar to the measurement sample 4A except that the RT-LAMP reaction mixture A was changed to the RT-LAMP reaction mixture B.

A measurement sample 5A was prepared in a manner similar to the measurement sample 3A except that the addition amount of mRNA of CK19 was changed to 2.5×10⁴ (copies/reaction).

A measurement sample 5B was prepared in a manner similar to the measurement sample 5A except that the RT-LAMP reaction mixture A was changed to the RT-LAMP reaction mixture B.

Schematic compositions of the measurement samples are shown in Table 4.

TABLE 4

| | | | Nucleic | |
|---|---|---|---|---|
| | | | acid | |
| | | | addition | |
| | | Nucleic | amount | RT-LAMP |
| | Model | acid | (copies/ | reaction |
| | sample | (mRNA) | reaction) | mixture |
| Measurement | contained | RBCS-1A | 1.5 × 10⁸ | A (for CK19 |
| sample 3A | | CK19 | 2.5 × 10⁵ | measurement) |
| Measurement | contained | RBCS-1A | 1.5 × 10⁸ | B (for |
| sample 3B | | CK19 | 2.5 × 10⁵ | RBCS-1A |
| | | | | measurement) |
| Measurement | contained | RBCS-1A | 1.5 × 10⁸ | A (for CK19 |
| sample 4A | | CK19 | 2.5 × 10⁶ | measurement) |
| Measurement | not | RBCS-1A | 1.5 × 10⁸ | B (for |
| sample 4B | contained | CK19 | 2.5 × 10⁶ | RBCS-1A |
| | | | | measurement) |
| Measurement | contained | RBCS-1A | 1.5 × 10⁸ | A (for CK19 |
| sample 5A | | CK-19 | 2.5 × 10⁴ | measurement) |
| Measurement | contained | RBCS-1A | 1.5 × 10⁸ | B (for |
| sample 5B | | CK-19 | 2.5 × 10⁴ | RBCS-1A |
| | | | | measurement) |

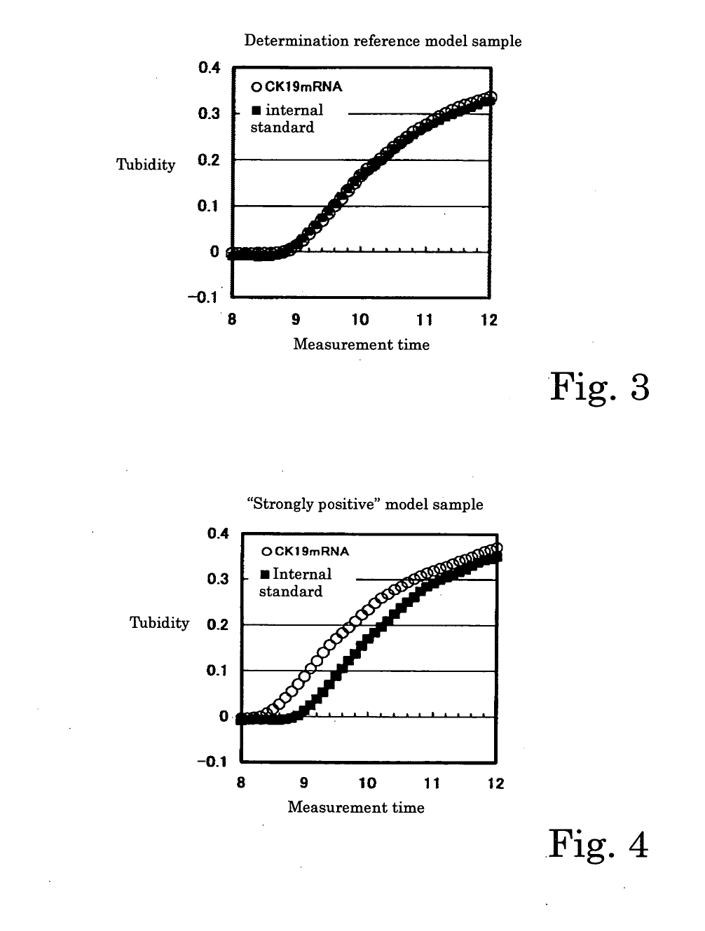

With LA-200 from TERAMECS Co., Ltd., cDNA corresponding to mRNA contained in each of the measurement samples was amplified by RT-LAMP and turbidity changes in each of the measurement samples were measured. Measurement results are shown in FIGS. 3 to 5.

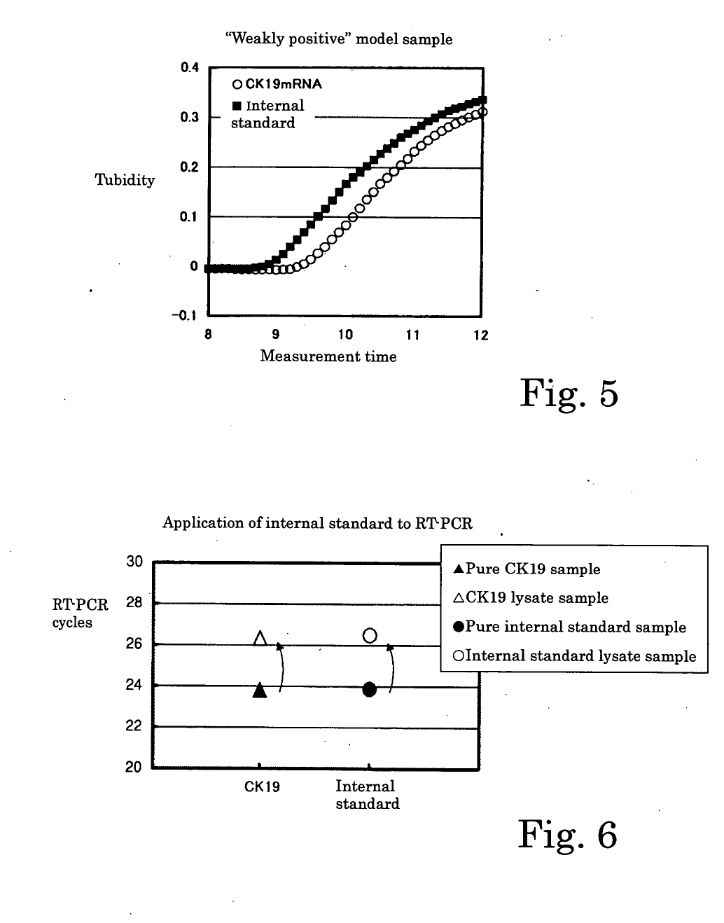

The case where the expression amount of mRNA of CK19 is 2.5×10⁵ (copies/reaction) was used as a reference (threshold) for determining whether cancer metastasis to lymph nodes is strongly positive or weakly positive. FIG. 3 shows results of measurement of the measurement sample 3A (2.5×10⁵ (copies/reaction) of mRNA of CK19) and the measurement sample 3B (1.5×10⁸ (copies/reaction) of the internal standard nucleic acid (mRNA of RBCS-1A). In FIG. 3, the turbidity change of the internal standard nucleic acid overlaps the turbidity change of mRNA of CK19 of 2.5×10⁵ (copies/reaction) as a determination reference. Consequently, it is understood that the internal standard nucleic acid has the same amplification efficiency as that of the determination reference of mRNA of CK19 even when there is the influence of the amplification inhibitor, and 1.5×10⁸ (copies/reaction) of the internal standard nucleic acid corresponds to 2.5×10⁵ (copies/reaction) of mRNA of CK19. FIG. 4 shows results of measurement of the measurement sample 4A containing mRNA of CK19 of the amount corresponding to “strongly positive”, and the measurement sample 4B containing 1.5×10⁸ (copies/reaction) of the internal standard nucleic acid. It is understood that turbidity of CK19 increases faster than the internal standard nucleic acid. FIG. 5 shows results of measurement of the measurement sample 5A containing mRNA of CK19 of the amount corresponding to “weakly positive”, and the measurement sample 5B containing 1.5×10⁸ (copies/reaction) of the internal standard nucleic acid. It is understood that turbidity of CK19 increases slower than the internal standard nucleic acid. From the above, it can be determined that the expression amount of the target nucleic acid is larger than that of the internal standard nucleic acid as a reference or not.

### EXAMPLE 4

Preparation of Reference Measurement Sample

A solution obtained by adding 2.5×10⁵ (copies/reaction) of mRNA of CK19 to 2 μl of a solubilization reagent was prepared. The solution was added to 23 μl of the RT-LAMP reaction mixture A prepared in Example 1, thereby preparing a reference measurement sample 1. The expression amount of 2.5×10⁵ (copies/reaction) of mRNA of CK19 is a reference value for determining whether metastasis of cancer to a lymph node is strongly positive (++) or weakly positive (+).

A solution obtained by adding 2.5×10⁴ (copies/reaction) of mRNA of CK19 to 2 μl of a solubilization reagent was prepared. The solution was added to 23 μl of the RT-LAMP reaction mixture A, thereby preparing a reference measurement sample 2. The expression amount of 2.5×10⁴ (copies/reaction) of mRNA of CK19 is a reference value for determining whether metastasis of cancer to a lymph node is weakly positive (+) or negative (−).

A solution obtained by adding 1.5×10⁸ (copies/reaction) of the internal standard nucleic acid (mRNA of RBCS-1A) to 2 μl of a solubilization reagent was prepared. The solution was added to 23 μl of the RT-LAMP reaction mixture B, thereby preparing a measurement sample IC (internal standard control). As described in Example 3, the expression amount of 1.5×10⁸ (copies/reaction) of the internal standard nucleic acid corresponds to that of 2.5×10⁵ of mRNA of CK19, and is a reference value for determining whether metastasis of cancer to a lymph node is weakly positive (+) or negative (−).

With LA-200 from TERAMECS Co., Ltd., cDNA corresponding to mRNA contained in each of the measurement samples was amplified by the RT-LAMP and time required until turbidity reaches 0.1 were measured. Measurement results are shown in Table 5.

TABLE 5

| | | Nucleic | | |
|---|---|---|---|---|
| | | acid | | |
| | | addition | | |
| | Nucleic | amount | RT-LAMP | Detection |
| | acid | (copies/ | reaction | time |
| | (mRNA) | reaction) | mixture | (minutes) |
| Reference | CK19 | 2.5 × 10⁵ | A | 10.6 |
| measurement | | | | |
| sample 1 | | | | |
| Reference | CK19 | 2.5 × 10⁴ | A | 11.4 |
| measurement | | | | |
| sample 2 | | | | |
| Measurement | RBCS-1A | 1.5 × 10⁸ | B | 11.4 |
| sample IC | | | | |

In the measurement results, a measurement value (detection time) CK19_{C1} as a reference of determining whether CK19 is positive/weakly positive (++/+) in the absence of the amplification inhibitor is 10.6 minutes, and a measurement value (detection time) CK19_{C2} as a reference of determining metastasis is weakly positive/negative (+/−) is 11.4 minutes. A measurement value IC_{C} as a reference of the internal standard is 11.4 minutes.

Preparation of Measurement Samples

Preparation of Nucleic Acid Containing Samples E to G

4 ml of a solubilization reagent was added to a human lymph node E with positive cancer metastasis isolated by a breast cancer operation and the resultant was homogenized at 12,000 rpm by a metal blender, thereby preparing a nucleic acid containing sample E. Similarly, nucleic acid containing samples F and G were prepared from human lymph nodes F and G with positive cancer metastasis removed during a breast cancer operation.

Preparation of Measurement Samples E1 to G1

2 μl of a solution obtained by adding 1.5×10⁸ (copies/reaction) of mRNA of RBCS-1A as an internal standard nucleic acid to the nucleic acid containing sample E and mixing the resultant for 30 seconds by a vortex mixer was added to 23 μl of an RT-LAMP reaction mixture A (RT-LAMP reaction mixture for measuring CK19), thereby preparing a measurement sample E1.

In a manner similar to the preparation of the measurement sample E1 except that the nucleic acid containing samples F and G were used in place of the nucleic acid containing sample E, measurement samples F1 and G1 were prepared.

Preparation of Measurement Samples E2 to G2

In a manner similar to the preparation of the measurement samples E1 to G1 except that an RT-LAMP reaction mixture B (RT-LAMP reaction mixture for measuring RBCS-1A) was used in place of the RT-LAMP reaction mixture A, measurement samples E2 to G2 were prepared.

Schematic compositions of the measurement samples are shown in Table 6.

TABLE 6

| | | Nucleic acid | RT-LAMP |
|---|---|---|---|
| | | containing | reaction |
| | Sample | sample | mixture |
| Measurement | E | E | A (for CK19 |
| sample E1 | | | measurement) |
| Measurement | F | F | A (for CK19 |
| sample F1 | | | measurement) |
| Measurement | G | G | A (for CK19 |
| sample G1 | | | measurement) |
| Measurement | E | E | B (for RBCS-1A |
| sample E2 | | | measurement) |
| Measurement | F | F | B (for RBCS-1A |
| sample F2 | | | measurement) |
| Measurement | G | G | B (for RBCS-1A |
| sample G2 | | | measurement) |

With LA-200 from TERAMECS Co., Ltd., cDNA corresponding to mRNA contained in each of the measurement samples was amplified by the RT-LAMP and time (detection time) required until turbidity of each of the measurement samples reaches 0.1 were measured. Measurement results are shown in Table 7.

TABLE 7

| | Internal | | | | | | | | | |
|---|---|---|---|---|---|---|---|---|---|---|
| | standard | | Internal | | | | | | | |
| | nucleic | | standard | Detection | | | CK19 | CK19 | Deter- | Deter- |
| | acid | CK19 | control | time | CK19 | CK19 | corrected | corrected | mination | mination |
| | detection | detection | detection | difference | determination | determination | determination | determination | result | result |
| | time ICS | time | time ICC | ICS − ICC | reference | reference | reference | reference | before | after |
| | (minutes) | (minutes) | (minutes) | (minutes) | ++/+ | +/− | ++/− | +/− | correction | correction |
| Sample E | 14.7 | 13.7 | 11.4 | 3.3 | 10.6 | 11.4 | 13.9 | 14.7 | − | ++ |
| Sample F | 12.2 | 11.5 | 11.4 | 0.8 | 10.6 | 11.4 | 11.4 | 12.2 | − | + |
| Sample G | 12.6 | 12.2 | 11.4 | 1.2 | 10.6 | 11.4 | 11.8 | 12.6 | − | + |

As shown in Table 7, in the case of the sample E, detection time of the internal standard nucleic acid in the measurement sample is 14.7 minutes, and detection time of CK19 is 13.7 minutes. As described above, the measurement value (detection time) as a reference of determination of whether CK19 is positive/weakly positive (++/+) in the absence of an amplification inhibitor is 10.6 minutes, and the measurement value (detection time) as a reference of determination whether metastasis is weakly positive/negative (+/−) is 11.4 minutes. If the determination reference is applied as it is to a measurement value of CK19 of the sample E, the determination result is negative (−). However, there is the difference between the detection time of 14.7 minutes of the internal standard nucleic acid of the sample E and the detection time of 11.4 minutes (detection time in the absence of the amplification inhibitor) in the internal standard control is 3.3 minutes. The detection time difference is the influence of the nucleic acid amplification inhabitation by the amplification inhibitor. Therefore, on the basis of the detection time difference, the reference value of determination of whether CK19 is positive/weakly positive (++/+) is corrected to 13.9 minutes, and the reference value of determination of whether CK19 is weakly positive/negative (+/−) is corrected to 14.7 minutes. Consequently, the determination result becomes positive (++), and correct determination in which the influence of the amplification inhibitor is corrected can be made. Also with respect to the samples F and G, similarly, a correct determination result corrected by using the internal standard nucleic acid could be obtained.

In Example 4, the reference value of determining the target nucleic acid is corrected on the basis of the detection time difference of the internal standard nucleic acid. Alternately, the detection value of the target nucleic acid in a sample may be corrected on the basis of the detection time difference of the internal standard nucleic acid. For example, in the case of the sample E, the CK19 detection time of 13.7 minutes is corrected to 10.4 minutes on the basis of the detection time difference of 3.3 minutes of the internal standard nucleic acid. By comparing the CK19 detection time of 10.4 minutes corrected in such a manner with 10.6 minutes as the reference value of determining whether CK19 is positive/weakly positive (++/+) and 11.4 minutes as the reference value of determining whether CK19 is weakly positive/negative (+/−), it can be determined that the result is positive (++)

### EXAMPLE 5

RT-PCR was carried out by using TaqMan® One-step RT-PCR Master Mix Reagents Kit of Applied Biosystems and a real-time quantitative PCR apparatus (ABI PRISM® 7700). TaqMan® One-step RT-PCR Master Mix Reagents Kit is a reagents kit for RT-PCR containing master mix (2×) and RNase amplification inhibitor mix (40×). ABI PRISM® 7700 performs nucleic acid amplification reaction with preset temperature and time and detects fluorescence intensity which increases in correspondence with amplification of the nucleic acid, thereby enabling the amplified nucleic acid to be determined.

By adding 1×10⁶ (copies/reaction) of mRNA of CK19 to the solubilization reagent prepared in Example 1, a sample 1 for nucleic acid amplification reaction was prepared. By adding 1×10⁶ (copies/reaction) of mRNA of CK19 to the model sample X prepared in Example 1, a sample 2 for nucleic acid amplification reaction was prepared. By adding 1×10⁶ (copies/reaction) of mRNA of RBCS-1A to the solubilization reagent, a sample 3 for nucleic acid amplification reaction was prepared. By adding 1×10⁶ (copies/reaction) of mRNA of RBCS-1A to the model sample X, a sample 4 for nucleic acid amplification reaction was prepared.

Subsequently, a reaction mixture containing master mix, RNase amplification inhibitor mix, and three kinds of primers was prepared. The reaction mixture 1 for CK19 contains 1× master mix, 1×RNase amplification inhibitor mix, 300 nM-forward primer (sequence number 15: cagatcgaag gcctgaagga), 300 nM reverse primer (sequence number 16: cttggcccct cagcgtact), and 200 nM TaqMan® probe (sequence number 17: gcctacctga agaagaacca tgaggaggaa). The reaction mixture 2 for RBCS-1A contains 1× master mix, 1×RNase amplification inhibitor mix, 300 nM forward primer (sequence number 18: cgcaaggctaacaacgacatt), 300 nM reverse primer (sequence number 19: ggccacacctgcatgca), and 200 nM TaqMan® probe (sequence number 20: ttccatcacaagcaacggcgga).

Next, the reaction mixture 1 and the sample 1 for nucleic acid amplification reaction (or the sample 2 for nucleic acid amplification reaction) were mixed, and RT-PCR was carried out by using the real-time quantitative PCR apparatus (ABI PRISM® 7700). In addition, the reaction mixture 2 and the sample 3 for nucleic acid amplification reaction (or the sample 4 for nucleic acid amplification reaction) were mixed, and RT-PCR was carried out by using the real-time quantitative PCR apparatus (ABI PRISM® 7700). The RT-PCR was executed by performing reverse transcription reaction at 48° C. for 30 minutes and, after that, performing 40 cycles of operation at 95° C. for 15 seconds and operation at 60° C. for one minute. FIG. 6 shows the results.

In FIG. 6, ▴ (pure CK19 sample) shows the result of measurement of amplification of a target nucleic acid by using the sample 1 for nucleic acid amplification reaction, that is, the sample containing no amplification inhibitor. Δ (CK19 solubilization sample) shows the result of measurement of amplification of a target nucleic acid by using the sample 2 for nucleic acid amplification reaction, that is, the sample containing the amplification inhibitor. From the results, it was found that amplification of the target nucleic acid delays about 2.5 cycles due to the influence of the amplification inhibitor. In FIG. 6, ● (pure internal standard sample) shows the result of measurement of amplification of an internal standard nucleic acid by using the sample 3 for nucleic acid amplification reaction, that is, the sample containing no amplification inhibitor. O (internal standard solubilization sample) shows the result of measurement of amplification of an internal standard nucleic acid by using the sample 4 for nucleic acid amplification reaction, that is, the sample containing no amplification inhibitor. From the results, it was found that amplification of the internal standard nucleic acid delays about 2.5 cycles due to the influence of the amplification inhibitor, and the influence of the amplification inhibitor on the internal standard nucleic acid is similar to that on the target nucleic acid. Therefore, it could be confirmed that the gene amplifying method such as RT-PCR can be applied to the present invention.

As described in the foregoing embodiments, the genetic testing method of the present invention can accurately and promptly obtain diagnosis support information useful for a doctor and the like to perform diagnosis even in the case where a measurement sample contains many substances inhibiting nucleic acid amplification, so that it can be used at a medical site.

The foregoing detailed description and examples have been provided by way of explanation and illustration, and are not intended to limit the scope of the appended claims. Many variations in the presently desirable embodiments illustrated herein will be obvious to one of ordinary skill in the art, and remain within the scope of the appended claims and their equivalents.

What is claimed is:

1. A genetic testing method for specifically amplifying a DNA complementary to a target nucleic acid existing in a biologic sample, measuring the amplified DNA, and generating diagnosis support information on the basis of a measurement result, comprising steps of:
(a) specifically amplifying a DNA complementary to an internal standard nucleic acid by using a known amount of the internal standard nucleic acid in the absence of the biologic sample component, and obtaining a first measurement result on the basis of the amplification, wherein the DNA has a property to be specifically amplified in the presence of the biologic sample component;
(b) specifically amplifying a DNA complementary to the internal standard nucleic acid by using an amount of a nucleic acid having a sequence identical to a sequence of the internal standard nucleic acid in the presence of the biologic sample component, and obtaining a second measurement result on the basis of the amplification, wherein the amount of the nucleic acid is the same as the known amount of the internal standard nucleic acid to be used in the step (a);
(c) specifically amplifying the DNA complementary to the target nucleic acid in the presence of the biologic sample component, and obtaining a third measurement result on the basis of the amplification;
(d) obtaining diagnosis support information on the basis of the third measurement result and a predetermined reference value; and
(e) determining influence on the diagnosis support information of an amplification inhibitor in the biologic sample component on the basis of the first and second measurement results.

2. The method of claim 1, wherein the difference between the first and second measurement results is compared with a threshold and, when the difference is larger than the threshold, it is determined that the influence of the amplification inhibitor is large.

3. The method of claim 2, wherein when it is determined that the influence of the amplification inhibitor is large, a warning about reliability of the diagnosis support information is issued.

4. The method of claim 2, wherein when it is determined that the influence of the amplification inhibitor is large, amplification of the target nucleic acid in the step (c) is performed and measured again under condition that the biologic sample is diluted at a predetermined dilution factor.

5. The method of claim 2, wherein when it is determined that the influence of the amplification inhibitor is large, the third measurement result or the reference value is corrected on the basis of the difference between the first and second measurement results.

6. The method of claim 1, wherein the diagnosis support information is a result of determination of whether the target nucleic acid amount contained in the biologic sample is larger than a reference target nucleic acid amount or not.

7. The method of claim 1, wherein the diagnosis support information is a result of determination that the biologic sample is positive or not, or the biologic sample is normal or abnormal.

8. The method of claim 1, wherein diagnosis support information is obtained on the basis of the third measurement result and first and second reference values.

9. The method of claim 8, wherein the diagnosis support information is a result of determination that the biologic sample is positive, weakly positive, or negative.

10. The method of claim 1, wherein the steps (a), (b) and (c) are performed under condition that the biologic sample is diluted at a predetermined dilution factor.

11. The method of claim 1, wherein the first measurement result is time when a first measurement value of measurement of amplification in the step (a) becomes a predetermined value, the second measurement result is time when a second measurement value of measurement of amplification in the step (b) becomes a predetermined value, and the third measurement result is time when a third measurement value of measurement of amplification in the step (c) becomes a predetermined value.

12. The method of claim 1, wherein the internal standard nucleic acid is a nucleic acid in which degree of amplification inhibition by an amplification inhibitor to the amplification of the DNA complementary to the internal standard nucleic acid is substantially the same as degree of amplification inhibition to the amplification of the DNA complementary to the target nucleic acid by the amplification inhibitor.

13. The method of claim 1, wherein the internal standard nucleic acid is a nucleic acid in which a relation between degree of amplification inhibition by an amplification inhibitor to the amplification of the DNA complementary to the internal standard nucleic acid and degree of amplification inhibition to the amplification of the DNA complementary to the target nucleic acid by the amplification inhibitor is known.

14. The method of claim 1, wherein the biologic sample is a lymph node, and the diagnosis support information is a result of determination of whether metastasis of cancer to a lymph node is positive or not.

15. A genetic testing method for specifically amplifying a DNA complementary to a target nucleic acid existing in a biologic sample, measuring the amplified DNA, and generating diagnosis support information on the basis of a measurement result, comprising steps of:
(a) specifically amplifying a DNA complementary to an internal standard nucleic acid by using a known amount of the internal standard nucleic acid in the absence of the biologic sample component, and obtaining a first measurement result on the basis of the amplification, wherein the DNA has a property to be specifically amplified in the presence of the biologic sample component;
(b) specifically amplifying a DNA complementary to the internal standard nucleic acid by using an amount of a nucleic acid having a sequence identical to a sequence of the internal standard nucleic acid in the presence of the biologic sample component, and obtaining a second measurement result on the basis of the amplification, wherein the amount of the nucleic acid is the same as the known amount of the internal standard nucleic acid to be used in the step (a);
(c) specifically amplifying the DNA complementary to the target nucleic acid in the presence of the biologic sample component, and obtaining a third measurement result on the basis of the amplification;
(d) correcting either the third measurement result or a reference value corresponding to a reference target nucleic acid amount on the basis of first and second measurement results; and
(e) obtaining diagnosis support information on the basis of the corrected one of the third measurement result and the reference value, and the other.

16. The method of claim 15, wherein the third measurement result is corrected, the corrected third measurement result is compared with the reference value, and genetic diagnosis is conducted.

17. The method of claim 15, wherein the reference value is corrected, the corrected reference value is compared with the third measurement result, and genetic diagnosis is conducted.

18. The method of claim 15, wherein the reference value is a first reference value corresponding to a first reference target nucleic acid amount and a second reference value corresponding to a second reference target nucleic acid amount.

19. The method of claim 15, wherein either the third measurement result or the reference value is corrected on the basis of the difference between the first and second measurement results.

20. A genetic testing method for specifically amplifying a DNA complementary to a target nucleic acid existing in a biologic sample, measuring the amplified DNA, and generating diagnosis support information on the basis of a measurement result, comprising steps of:
(a) specifically amplifying a DNA complementary to an internal standard nucleic acid by using a known amount of the internal standard nucleic acid in the absence of the biologic sample component, and obtaining a first measurement result on the basis of the amplification, wherein the DNA has a property to be specifically amplified in the presence of the biologic sample component;
(b) specifically amplifying the DNA complementary to the target nucleic acid in the presence of the biologic sample component, and obtaining a second measurement result on the basis of the amplification; and
(c) comparing the first and second measurement results to obtain diagnosis support information.


SEQUENCE LISTING (20 sequences)

SEQ ID NO: 1 - DNA, 1360 bp, Homo sapiens
cgggggttgctccgtccgtgctccgcctcgccatgacttcctacagctatcgccagtcgtcggccacgtcgtccttcggaggcctgggcggcggctccgtgcgttttgggccgggggtcgcttttcgcgcgcccagcattcacgggggctccggcggccgcggcgtatccgtgtcctccgcccgctttgtgtcctcgtcctcctcggggggctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaacgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctggcggatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaaggg

SEQ ID NO: 2 - DNA, 878 bp, Arabidopsis sp.
tcagtcacacaaagagtaaagaagaacaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccgaattggctaaggaagttgactaccttatccgcaacaagtggattccttgtgttgaattcgagttggagcacggatttgtgtaccgtgagcacggtaactcacccggatactatgatggacggtactggacaatgtggaagcttcccttgttcggttgcaccgactccgctcaagtgttgaaggaagtggaagagtgcaagaaggagtaccccaatgccttcattaggatcatcggattcgacaacacccgtcaagtccagtgcatcagtttcattgcctacaagccaccaagcttcaccggttaatttccctttgcttttgtgtaaacctcaaaactttatcccccatctttgattttatcccttgtttttctgcttttttcttctttcttgggttttaatttccggacttaacgtttgttttccggtttgcgagacatattctatcggattctcaactgtctgatgaaataaatatgtaatgttctataagtctttcaatttgatatgcatatcaacaaaaagaaaataggacaatgcggctacaaatatgaaatttacaagtttaagaaccatgagtcgctaaagaaatcattaagaaaattagtttcac

SEQ ID NO: 3 - DNA, 41 bp, Artificial Sequence
  chemically synthesized nucleic acid amplification primer
ggagttctcaatggtggcaccaactactacacgaccatcca

SEQ ID NO: 4 - DNA, 41 bp, Artificial Sequence
  chemically synthesized nucleic acid amplification primer
gtcctgcagatcgacaacgcctccgtctcaaacttggttcg

SEQ ID NO: 5 - DNA, 18 bp, Artificial Sequence
  chemically synthesized nucleic acid amplification primer
tggtaccagaagcagggg

SEQ ID NO: 6 - DNA, 19 bp, Artificial Sequence
  chemically synthesized nucleic acid amplification primer
gttgatgtcggcctccacg

SEQ ID NO: 7 - DNA, 18 bp, Artificial Sequence
  chemically synthesized nucleic acid amplification primer
agaatcttgtcccgcagg

SEQ ID NO: 8 - DNA, 17 bp, Artificial Sequence
  chemically synthesized nucleic acid amplification primer
cgtctggctgcagatga

SEQ ID NO: 9 - DNA, 41 bp, Artificial Sequence
  chemically synthesized nucleic acid amplification primer
accgaacaagggaagcttccactgagcacggtaactcaccc

SEQ ID NO: 10 - DNA, 41 bp, Artificial Sequence
  chemically synthesized nucleic acid amplification primer
accgactccgctcaagtgttgtcctaatgaaggcattgggg

SEQ ID NO: 11 - DNA, 20 bp, Artificial Sequence
  chemically synthesized nucleic acid amplification primer
tggagcacggatttgtgtac

SEQ ID NO: 12 - DNA, 18 bp, Artificial Sequence
  chemically synthesized nucleic acid amplification primer
cactggacttggcgggtg

SEQ ID NO: 13 - DNA, 19 bp, Artificial Sequence
  chemically synthesized nucleic acid amplification primer
ccagtaccgtccatcatag

SEQ ID NO: 14 - DNA, 20 bp, Artificial Sequence
  chemically synthesized nucleic acid amplification primer
gaagtggaagagtgcaagaa

SEQ ID NO: 15 - DNA, 20 bp, Artificial Sequence
  chemically synthesized nucleic acid amplification primer
cagatcgaaggcctgaagga

SEQ ID NO: 16 - DNA, 19 bp, Artificial Sequence
  chemically synthesized nucleic acid amplification primer
cttggcccctcagcgtact

SEQ ID NO: 17 - DNA, 30 bp, Artificial Sequence
  chemically synthesized nucleic acid amplification primer
gcctacctgaagaagaaccatgaggaggaa

SEQ ID NO: 18 - DNA, 21 bp, Artificial Sequence
  chemically synthesized nucleic acid amplification primer
cgcaaggctaacaacgacatt

SEQ ID NO: 19 - DNA, 17 bp, Artificial Sequence
  chemically synthesized nucleic acid amplification primer
ggccacacctgcatgca

SEQ ID NO: 20 - DNA, 22 bp, Artificial Sequence
  chemically synthesized nucleic acid amplification primer
ttccatcacaagcaacggcgga